(19) United States
(12) Patent Application Publication
(10) Pub. No.: US 2024/0293075 A1
(43) Pub. Date: Sep. 5, 2024

(54) EAR-WORN DEVICES INCLUDING DIAGNOSTICS AND MONITORING FEATURES FOR DENTAL HEALTH

(71) Applicant: Starkey Laboratories, Inc., Eden Prairie, MN (US)

(72) Inventors: Paul Shriner, Hopkins, MN (US); Gregory John Haubrich, Champlin, MN (US); Matan Sivan, Tel Aviv (IL); Yoshi Kasahara, St. Louis Park, MN (US)

(21) Appl. No.: 18/587,564

(22) Filed: Feb. 26, 2024

Related U.S. Application Data
(60) Provisional application No. 63/449,097, filed on Mar. 1, 2023.

(51) Int. Cl. A61B 5/00; A61B 5/08

(52) U.S. Cl. CPC A61B 5/4557; A61B 5/0803; A61B 5/0816; A61B 5/0826; A61B 5/4809; A61B 5/6803; A61B 5/746

(57) ABSTRACT

Embodiments herein relate to ear-wearable device systems with features to monitor dental health. In an embodiment, an ear-wearable device system is included having an ear-wearable device. The ear-wearable device can include a microphone and a sensor package. The sensor package can include a motion sensor. The ear-wearable device system can be configured to evaluate signals from at least one of the microphone and the motion sensor to detect an occurrence of a dental health condition. In some embodiments, an ear-wearable device system herein can be configured to compare signals from at least one of a microphone and a motion sensor from a time period prior to a dental procedure with signals from a time period after the dental procedure. In some embodiments the ear-wearable device system is configured to evaluate signals a sensor to detect an occurrence of a dental hygiene event. Other embodiments are also included herein.

This application claims the benefit of U.S. Provisional Application No. 63/449,097 filed Mar. 1, 2023, the content of which is herein incorporated by reference in its entirety.

### FIELD

Embodiments herein relate to ear-wearable devices and ear-wearable device systems with features to monitor dental health.

### BACKGROUND

Dental health affects the ability of individuals to eat, swallow, speak, and participate normally in many daily activities of life. Dental health affects a person's self-esteem, school performance, work performance, and social activities.

Dental health issues cause pain and disability for millions each year. Dental health issues can include cavities (or tooth decay), tooth loss, gum disease, bruxism, temporomandibular joint (TMJ) issues, and the like. As one specific example, tooth decay is a common chronic disease that can cause pain, suffering, and diminished quality of life. Left untreated, tooth decay can progress and lead to infection, tooth loss, and more complex and expensive treatments.

In some cases, individuals may experience a sudden onset of mouth pain or discomfort signaling that there is a dental health issue. However, in other cases, some individuals may not notice that they have problems at all as they get used to the feel of their mouth with issues and/or depending on their cognitive state. Regardless, it can be important to address dental health issues early and proactively for both optimization of outcomes as well as minimization of cost.

### SUMMARY

Embodiments herein relate to ear-wearable devices and ear-wearable device systems with features to monitor dental health. In a first aspect, an ear-wearable device system can be included having an ear-wearable device. The ear-wearable device can include a control circuit and a microphone in electrical communication with the control circuit. The ear-wearable device can include a sensor package in electrical communication with the control circuit. The sensor package can include a motion sensor. The ear-wearable device system can be configured to evaluate signals from at least one of the microphone and the motion sensor to detect an occurrence of a dental health condition.

In a second aspect, in addition to one or more of the preceding or following aspects, or in the alternative to some aspects, the dental health condition can include at least one selected from the group consisting of bruxism, a TMJ joint issue, oral pain, and tooth decay.

In a third aspect, in addition to one or more of the preceding or following aspects, or in the alternative to some aspects, the ear-wearable device can be a hearing-assistance device.

In a fourth aspect, in addition to one or more of the preceding or following aspects, or in the alternative to some aspects, the ear-wearable device system can be configured to issue an alert or notification regarding detected occurrences of the dental health condition.

In a fifth aspect, in addition to one or more of the preceding or following aspects, or in the alternative to some aspects, the ear-wearable device can be configured to time stamp detected occurrences of dental health related conditions and identify time-related patterns of the same.

In a sixth aspect, in addition to one or more of the preceding or following aspects, or in the alternative to some aspects, the ear-wearable device can be configured to identify sleep bruxism.

In a seventh aspect, in addition to one or more of the preceding or following aspects, or in the alternative to some aspects, the ear-wearable device system can be configured to distinguish between awake bruxism and sleep bruxism.

In an eighth aspect, in addition to one or more of the preceding or following aspects, or in the alternative to some aspects, the ear-wearable device system can further include a second ear-wearable device. The second ear-wearable device can include a second control circuit, a second microphone, and a second sensor package. The second ear-wearable device can be configured to be wearable on an opposite ear versus the ear-wearable device.

In a ninth aspect, in addition to one or more of the preceding or following aspects, or in the alternative to some aspects, the ear-wearable device system can be configured to identify differences in dental health related conditions between a left side of the mouth and a right side of the mouth.

In a tenth aspect, in addition to one or more of the preceding or following aspects, or in the alternative to some aspects, the ear-wearable device system can be configured to identify an abnormal chewing motion.

In an eleventh aspect, in addition to one or more of the preceding or following aspects, or in the alternative to some aspects, the abnormal chewing motion can include an offset chewing motion.

In a twelfth aspect, in addition to one or more of the preceding or following aspects, or in the alternative to some aspects, the ear-wearable device system can be configured to identify differences in sounds originating from a left side of the mouth versus a right side of the mouth.

In a thirteenth aspect, in addition to one or more of the preceding or following aspects, or in the alternative to some aspects, the ear-wearable device system can be configured to distinguish between left sided TMJ joint problems and right sided TMJ joint problems.

In a fourteenth aspect, in addition to one or more of the preceding or following aspects, or in the alternative to some aspects, the ear-wearable device system can be further configured to detect abnormal breathing.

In a fifteenth aspect, in addition to one or more of the preceding or following aspects, or in the alternative to some aspects, the abnormal breathing can include at least one selected from the group consisting of snoring and sleep apnea.

In a sixteenth aspect, a method of detecting dental health conditions using an ear-wearable system is included. The method can include monitoring a device wearer with sensors of the ear-wearable system. The sensors can include at least one of a microphone and a motion sensor. The method can further include evaluating signals from at least one of the microphone and the motion sensor to detect an occurrence of a dental health condition and issuing an alert or notification regarding detected occurrences of the dental health condition.

In a seventeenth aspect, in addition to one or more of the preceding or following aspects, or in the alternative to some aspects, the dental health condition can include at least one selected from the group consisting of bruxism, a TMJ joint issue, oral pain, and tooth decay.

In an eighteenth aspect, in addition to one or more of the preceding or following aspects, or in the alternative to some aspects, can further include time stamping detected occurrences of dental health conditions and identifying time-related patterns of the same.

In a nineteenth aspect, in addition to one or more of the preceding or following aspects, or in the alternative to some aspects, the method can further include distinguishing between awake bruxism and sleep bruxism.

In a twentieth aspect, in addition to one or more of the preceding or following aspects, or in the alternative to some aspects, the method can further include identifying differences in dental health conditions between a left side of the mouth and a right side of the mouth.

In a twenty-first aspect, in addition to one or more of the preceding or following aspects, or in the alternative to some aspects, the method can further include identifying differences in sounds originating from a left side of the mouth versus a right side of the mouth.

In a twenty-second aspect, in addition to one or more of the preceding or following aspects, or in the alternative to some aspects, the method can further include distinguishing between left sided TMJ joint problems and right sided TMJ joint problems.

In a twenty-third aspect, in addition to one or more of the preceding or following aspects, or in the alternative to some aspects, the method can further include detecting abnormal breathing.

In a twenty-fourth aspect, in addition to one or more of the preceding or following aspects, or in the alternative to some aspects, the abnormal breathing can include at least one selected from the group consisting of snoring and sleep apnea.

In a twenty-fifth aspect, an ear-wearable device system can be included having an ear-wearable device. The ear-wearable device can include a control circuit and a microphone in electrical communication with the control circuit. The ear-wearable device can include a sensor package including a motion sensor. The sensor package can be in electrical communication with the control circuit. The ear-wearable device system can be configured to compare signals from at least one of the microphone and the motion sensor from a time period prior to a dental procedure with signals from at least one of the microphone and the motion sensor from a time period after the dental procedure.

In a twenty-sixth aspect, in addition to one or more of the preceding or following aspects, or in the alternative to some aspects, the dental procedure can include at least one selected from the group consisting of a dental implant, a crown, an orthodontic adjustment, and a tooth filling.

In a twenty-seventh aspect, in addition to one or more of the preceding or following aspects, or in the alternative to some aspects, the ear-wearable device system can be configured to receive an input regarding a type of dental procedure from a system user.

In a twenty-eighth aspect, in addition to one or more of the preceding or following aspects, or in the alternative to some aspects, the ear-wearable device system can be configured to issue an alert or notification when a worsening of a dental condition is detected based on the signals from at least one of the microphone and the motion sensor.

In a twenty-ninth aspect, in addition to one or more of the preceding or following aspects, or in the alternative to some aspects, the ear-wearable device can be a hearing-assistance device.

In a thirtieth aspect, in addition to one or more of the preceding or following aspects, or in the alternative to some aspects, the ear-wearable device system can further include a second ear-wearable device. The second ear-wearable device can include a second control circuit, a second microphone, and a second sensor package. The second ear-wearable device can be configured to be wearable on an opposite ear versus the ear-wearable device.

In a thirty-first aspect, in addition to one or more of the preceding or following aspects, or in the alternative to some aspects, the ear-wearable device system can be configured to identify differences in dental health related conditions between a left side of the mouth and a right side of the mouth.

In a thirty-second aspect, in addition to one or more of the preceding or following aspects, or in the alternative to some aspects, the ear-wearable device system can be configured to identify differences in sounds originating from a left side of the mouth versus a right side of the mouth.

In a thirty-third aspect, in addition to one or more of the preceding or following aspects, or in the alternative to some aspects, the ear-wearable device system can be configured to identify an abnormal chewing motion.

In a thirty-fourth aspect, in addition to one or more of the preceding or following aspects, or in the alternative to some aspects, the abnormal chewing motion can include an offset chewing motion.

In a thirty-fifth aspect, a method of tracking effects of a dental procedure can be included. The method can include receiving an input regarding a dental procedure and monitoring a device wearer with sensors of the ear-wearable system. The sensors can include at least one of a microphone and a motion sensor. The method can further include comparing signals from at least one of the microphone and the motion sensor from a time period prior to the dental procedure with signals from at least one of the microphone and the motion sensor from a time period after the dental procedure.

In a thirty-sixth aspect, in addition to one or more of the preceding or following aspects, or in the alternative to some aspects, the method can further include issuing an alert or notification when a worsening of a dental condition can be detected based on the signals from at least one of the microphone and the motion sensor.

In a thirty-seventh aspect, in addition to one or more of the preceding or following aspects, or in the alternative to some aspects, the method can further include identifying differences in dental health conditions between a left side of the mouth and a right side of the mouth.

In a thirty-eighth aspect, in addition to one or more of the preceding or following aspects, or in the alternative to some aspects, the method can further include identifying differences in sounds originating from a left side of the mouth versus a right side of the mouth.

In a thirty-ninth aspect, an ear-wearable device system can be included having an ear-wearable device. The ear-wearable device can include a control circuit, a microphone in electrical communication with the control circuit, and a sensor package in electrical communication with the control circuit. The sensor package can include a motion sensor in electrical communication with the control circuit. The ear-wearable device system can be configured to evaluate signals from at least one of the microphone and the motion sensor to detect an occurrence of a dental hygiene event.

In a fortieth aspect, in addition to one or more of the preceding or following aspects, or in the alternative to some aspects, the dental hygiene event can include at least one selected from the group consisting of teeth brushing and teeth flossing.

In a forty-first aspect, in addition to one or more of the preceding or following aspects, or in the alternative to some aspects, the ear-wearable device system can be configured to track occurrences of dental hygiene events over time.

In a forty-second aspect, in addition to one or more of the preceding or following aspects, or in the alternative to some aspects, the ear-wearable device system can be configured to track frequencies of dental hygiene events over time.

In a forty-third aspect, in addition to one or more of the preceding or following aspects, or in the alternative to some aspects, the ear-wearable device system can be configured to track durations of dental hygiene events over time.

In a forty-fourth aspect, in addition to one or more of the preceding or following aspects, or in the alternative to some aspects, the ear-wearable device can be configured to time stamp detected occurrences of dental hygiene events and identify time-related patterns of the same.

In a forty-fifth aspect, in addition to one or more of the preceding or following aspects, or in the alternative to some aspects, the ear-wearable device can be a hearing-assistance device.

In a forty-sixth aspect, in addition to one or more of the preceding or following aspects, or in the alternative to some aspects, the ear-wearable device system can further include a second ear-wearable device. The second ear-wearable device can include a second control circuit, a second microphone, and a second sensor package. The second ear-wearable device can be configured to be wearable on an opposite ear versus the ear-wearable device.

In a forty-seventh aspect, in addition to one or more of the preceding or following aspects, or in the alternative to some aspects, the ear-wearable device system can be configured to prompt a device wearer to undertake the dental hygiene event.

In a forty-eighth aspect, a method of detecting dental hygiene events can be included. The method can include monitoring a device wearer with sensors of the ear-wearable system, the sensors including at least one of a microphone and a motion sensor, and evaluating signals from at least one of the microphone and the motion sensor to detect an occurrence of a dental hygiene event.

In a forty-ninth aspect, in addition to one or more of the preceding or following aspects, or in the alternative to some aspects, the method can further include tracking occurrences of dental hygiene events over time.

In a fiftieth aspect, in addition to one or more of the preceding or following aspects, or in the alternative to some aspects, the method can further include tracking frequencies of dental hygiene events over time.

In a fifty-first aspect, in addition to one or more of the preceding or following aspects, or in the alternative to some aspects, the method can further include tracking durations of dental hygiene events over time.

In a fifty-second aspect, in addition to one or more of the preceding or following aspects, or in the alternative to some aspects, the method can further include timing stamp detected occurrences of dental hygiene events and identify time-related patterns of the same.

In a fifty-third aspect, in addition to one or more of the preceding or following aspects, or in the alternative to some aspects, the method can further include prompting the device wearer to undertake the dental hygiene event.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a device wearer along with components of an ear-wearable device system in accordance with various embodiments herein.

FIG. 2 is a schematic view of a device wearer along with components of an ear-wearable device system in accordance with various embodiments herein.

FIG. 3 is a schematic view of components of an ear-wearable device system in accordance with various embodiments herein.

FIG. 4 is a flowchart of operations performed in accordance with various embodiments herein.

FIG. 5 is a schematic view of components of an ear-wearable device system in accordance with various embodiments herein.

FIG. 6 is a schematic view of components of an ear-wearable device system in accordance with various embodiments herein.

FIG. 7 is a diagram of data processing operations in accordance with various embodiments herein.

FIG. 8 is a flowchart of operations performed in accordance with various embodiments herein.

FIG. 9 is a schematic view of components of an ear-wearable device system in accordance with various embodiments herein.

FIG. 10 is a block diagram of components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, dental health issues cause pain and disability for millions each year and are a substantial public health issue. In some cases, individuals may experience a sudden onset of mouth pain or discomfort signaling that there is a dental health issue but in other cases individuals may not notice that they have and/or are developing problems at all.

Ear-wearable device systems herein can evaluate signals from one or more sensors (such as at least one of the microphone and a motion sensor) and can be used to detect the occurrence or presence of various dental health conditions. For example, ear-wearable device systems can detect dental health conditions such as bruxism, a TMJ joint issue, oral pain, and tooth decay. Such conditions can be logged and reported on such as through alerts or notifications to a device wearer and/or a care provider or clinician for the device wearer facilitating the receipt of needed dental health care.

Beyond detecting dental health conditions, ear-wearable systems herein can be used to track a device wearer's recovery or progress made after a dental procedure has been performed. For example, in some cases the ear-wearable device system can compare sensor signals (such as from at least one of the microphone and the motion sensor) from a time period prior to a dental procedure with signals from a time period after the dental procedure in order to determine whether the device wearer is improving as they should with respect to dental health and/or to determine if they are encountering any issues that may arise after the dental procedure. Such dental procedures can include, but are not limited to, a dental implant, a crown, an orthodontic adjustment, and a tooth filling.

Proper preventative care and dental hygiene can reduce the incidence of dental health problems. However, various individuals may not keep up with recommended dental hygiene regimens. Ear-wearable systems herein can be used to evaluate signals from one or more sensors to detect an occurrence of a dental hygiene event such as brushing or flossing. Detected dental hygiene events can be logged and reported on such as through alerts or notifications to a device wearer and/or a care provider or clinician. In some cases, reminders for dental hygiene can be provided to the device wearer and/or a care provider.

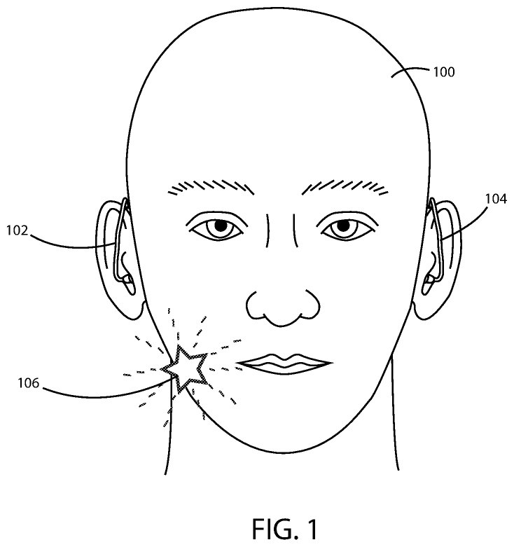

Referring now to FIG. 1, a schematic view of a device wearer 100 is shown along with components of an ear-wearable device system in accordance with various embodiments herein. The ear-wearable device system includes a first ear-wearable device 102 and a second ear-wearable device 104. The second ear-wearable device 104 can be configured to be wearable on an opposite ear versus an ear-wearable device 102.

However, it will be appreciated that in some embodiments only a single ear-wearable device may be included with the system.

In this example, the device wearer 100 is experiencing mouth discomfort or pain 106. Such mouth discomfort 106 can have various dental health causes including, but not limited to tooth decay, tooth damage, a TMJ joint issue, a gum issue, and the like. Alternatively, such mouth discomfort or pain 106 may relate to a dental procedure that has been performed such as a dental implant, a crown, an orthodontic adjustment, and a tooth filling.

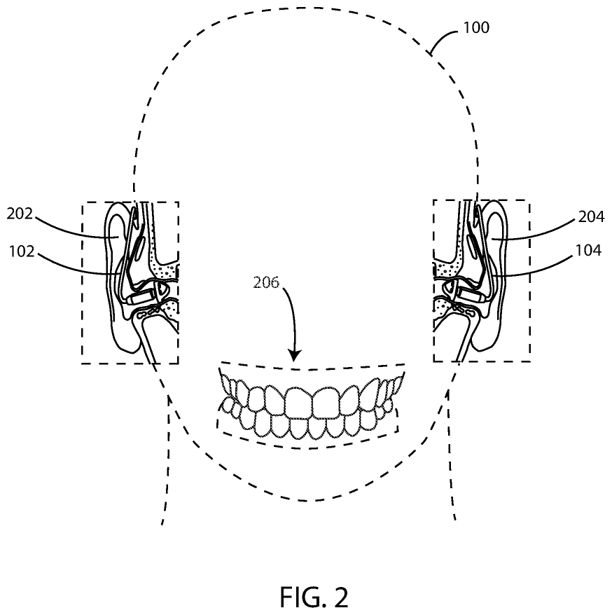

Referring now to FIG. 2, a schematic view is shown of a device wearer 100 along with components of an ear-wearable device system in accordance with various embodiments herein. The device wearer 100 is shown with their right ear 202 and left ear 204. The first ear-wearable device 102 can be worn on or about the right ear 202 and the second ear-wearable device 104 can be worn on or about the left ear 204. FIG. 2 also shows the mouth 206 of the device wearer 100. As the device wearer 100 talks, chews, swallows, etc. various movements and sounds coming from the mouth 206 area of the device wearer 100 can be detected with sensors herein. Signals from such sensors can be evaluated as described further herein to detect dental health conditions, track a device wearer's recovery and/or progress after a dental procedure, detect occurrences of dental hygiene events, and the like.

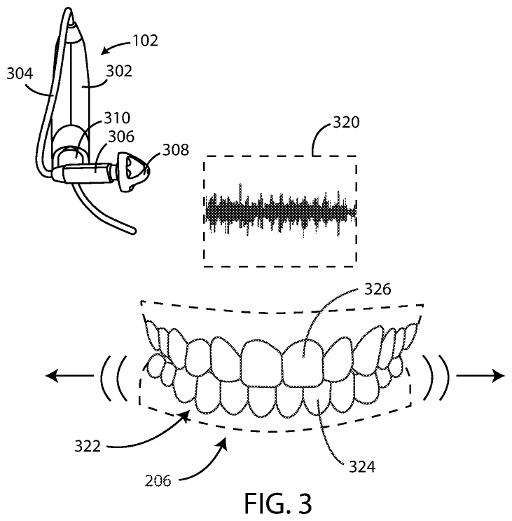

Referring now to FIG. 3, a schematic view of components of an ear-wearable device system is shown in accordance with various embodiments herein. The mouth 206 of a device wearer is shown along with lower teeth 324 on the jaw 322 and upper teeth 326.

As before, the ear-wearable device system includes an ear-wearable device 102. Many different types of ear-wearable devices are contemplated herein and as detailed more fully below. However, in this example the ear-wearable device 102 includes a housing 302, a cable 304, and a receiver 306. In this example, the ear-wearable device 102 also includes an ear bud 308 and a battery compartment 310. Sensor(s) of the ear-wearable device 102 can generate sensor signals 320.

Bruxism serves as an example of a dental health condition that is detectable with embodiments of systems herein. Bruxism is a condition characterized by the grinding and/or gnashing of teeth. As shown in FIG. 3, the lower teeth 324 can move with respect to the upper teeth 326, such as moving laterally and/or in other directions. Bruxism can lead to various negative outcomes including abnormal teeth wear, pain, stiffness, and disruption of sleep.

Systems and devices herein can be used to identify occurrences of bruxism. By way of example, bruxism causes certain characteristic sounds and/or movement patterns that are detectable using sensors that are part of car-wearable device systems herein. In some embodiments, systems and/or devices herein can use sensors to gather signal data and then analyze the same to identify the characteristic sounds and/or movement patterns or bruxism. Exemplary signal data processing techniques to identify specific conditions such as bruxism are described in greater detail below.

In various embodiments, the car-wearable device 102 can be configured to time stamp detected occurrences of dental health related conditions. The devices and/or systems herein can then evaluate the time stamped data and identify time-related patterns of the same. For example, the system can detect whether bruxism typically occurs at night. This is relevant as bruxism can be of two different types. Sleep bruxism, as implied by the name, occurs while an individual is sleeping. In contrast, awake bruxism occurs while an individual is awake. Systems and devices herein can be used to identify sleep bruxism. In various embodiments, the car-wearable device system can be configured to distinguish between awake bruxism and sleep bruxism. In some embodiments, the system can reference the time of day when an occurrence of bruxism is detected and if it occurs during normal sleeping hours then the system can take that as an indication that sleep bruxism has been detected. In contrast, if an occurrence of bruxism occurs during normal awake hours, then the system can take that as an indication that awake bruxism has been detected. In some embodiments, the system can evaluate sounds received through the microphone and/or movement from a motion sensor in order to detect sleep. In some embodiments, the system can detect sounds consistent with sounds made during sleep and conclude that the device wearer is sleeping. In some embodiments, the system can receive an input from another device or system to indicate that the device wearer is sleeping, such as an input from a sleep monitoring device. In some embodiments, the system can evaluate signals from a motion sensor and conclude that the individual is awake by observing movement consistent with an individual is awake. In some embodiments, a signal from a gyroscope can be evaluated to determine if the individual is lying down or in an upright position. In some embodiments, the system can conclude that the individual is likely sleeping if they are lying down and/or lying down during nighttime hours.

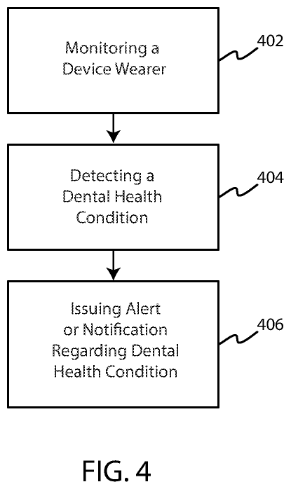

It will be appreciated that while FIG. 3 illustrates one type of car-wearable device (or car-wearable sensor device) consistent with embodiments herein, many other types of car-wearable devices are also contemplated. The term “ear-wearable device” as used herein can include devices that can aid a person with impaired hearing. The term “ear-wearable device” shall also refer to devices that can produce optimized or processed sound for persons with normal hearing. Ear-wearable devices herein can include hearing assistance devices. Ear-wearable devices herein can include, but are not limited to, behind-the-car (BTE), in-the car (ITE), in-the-canal (ITC), invisible-in-canal (IIC), receiver-in-canal (RIC), receiver in-the-car (RITE) and completely-in-the-canal (CIC) type hearing assistance devices. In some embodiments, the car-wearable device can be a hearing aid falling under 21 C.F.R. § 801.420. In some embodiments, the car-wearable device can be an over-the-counter (OTC) hearing aid. In another example, the car-wearable device can include one or more Personal Sound Amplification Products (PSAPs). In another example, the car-wearable device can include one or more cochlear implants, cochlear implant magnets, cochlear implant transducers, and cochlear implant processors. In another example, the hearing assistance device can include one or more “hearable” devices that provide various types of functionality. In other examples, car-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via the bone conduction pathway. In another example, the hearing assistance device can include an auditory brainstem implant, a cranial nerve (e.g., CN VIII) implant, and the like. Referring now to FIG. 4, a flowchart of operations performed is shown in accordance with various embodiments herein. In specific, FIG. 4 shows operations performed as part of a method of detecting dental health conditions using an car-wearable system. The set of operations performed can include monitoring 402 a device wearer with sensors of the ear-wearable system, the sensors including at least one of a microphone and a motion sensor. In some embodiments, monitoring can be performed continuously as car-wearable devices herein are worn. In some embodiments monitoring can be performed after receiving an input to turn on monitoring functions, such as an input received from the device wearer and/or a third party such as a care provider or a clinician. The set of operations can further include evaluating 404 signals from at least one of the microphone and the motion sensor to detect an occurrence of a dental health condition. Such detections can be performed by the system as described elsewhere herein regarding signal processing and/or pattern matching techniques. The set of operations can further include issuing an alert or notification regarding detected occurrences of a dental health condition 406. In some embodiments, the alert or notification can be directed to the device wearer. In some embodiments the alert or notification can be directed to a third party such as a care provider or a clinician.

While dental health issues may occur bilaterally, in many cases dental health issues may be unilateral such as a dental cavity or other tooth damage occurring on a particular tooth on the right or left side of the mouth. As another example, a TMJ joint issue may only occur with the TMJ joint on one side of the device wearer's jaw (right or left). Embodiments herein can be used to distinguish between dental health issues occurring on one side of the mouth or the other. Further, in some cases, asymmetries of sound or motion can be used to identify that a dental health issue is present. For example, pain on one side of the mouth may cause an individual to alter their chewing movements to favor one side and/or prevent contact on a side that may otherwise exacerbate pain. As such, embodiments herein can also be used to identify asymmetries in sound or motion between the two sides of the mouth in order to identify the presence of a dental health issue as well as distinguish between issues occurring on one side of the mouth versus the other.

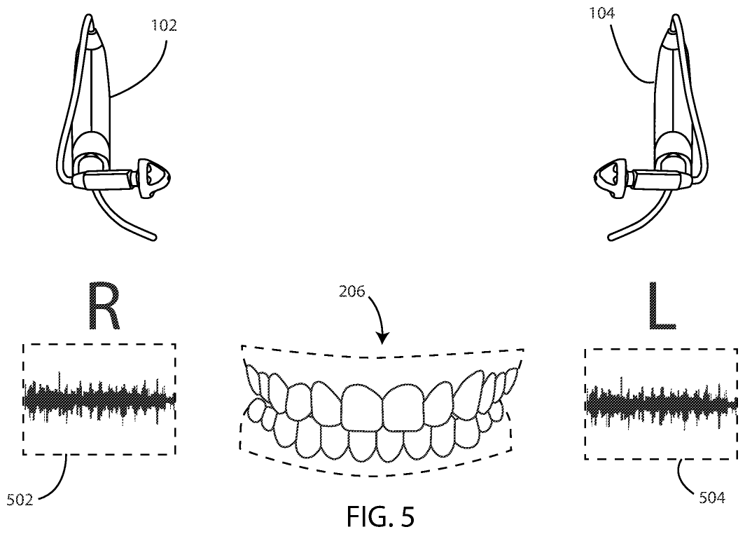

Referring now to FIG. 5, a schematic view of components of an car-wearable device system is shown in accordance with various embodiments herein. The mouth 206 of a device wearer is shown along with a first car-wearable device 102 and a second car-wearable device 104. Sensors of the first ear-wearable device can generate a right-side sensor signal 502 while sensors of the second car-wearable device 104 can generate a left-side sensor signal 504. Signals can be disambiguated with regard to whether they relate to the right side or left side using various techniques. In some embodiments, intensity differences between signals as gathered from an ear-wearable device on the right side versus the left side can be used to determine if the signal relates to the right side of the mouth versus the left side of the mouth. In some embodiments, timing differences between signals as gathered from an car-wearable device on the right side versus the left side can be used to determine if the signal relates to the right side of the mouth versus the left side of the mouth. In some embodiments, sensors can be configured and/or tuned to be sensitive to sound and/or motion coming from a particular direction. For example, directional microphones can be used to distinguish between sounds originating on the left side versus the right side. Other techniques of determining an origin location or region for sounds and/or motion are described in greater detail below. Thus, in various embodiments the car-wearable device system can be configured to identify differences in sounds originating from a left side of the mouth versus a right side of the mouth.

In various embodiments, the car-wearable device system can be configured to identify differences in dental health related conditions between a left side of the mouth 206 and a right side of the mouth 206 by evaluating and comparing the right-side sensor signals 502 and the left-side sensor signals 504. In some embodiments, right-side sensor signals 502 can be greater in magnitude than left-side sensor signals 504 if the dental health condition is occurring on the right side. For example, sounds consistent with popping of the jaw (as may occur with a TMJ joint problem) may be more intense in the right-side sensor signals 502 if the TJM joint problem occurs on the right side. As such, in various embodiments the car-wearable device 102 system can be configured to distinguish between left sided TMJ joint problems and right sided TMJ joint problems.

In various embodiments, the car-wearable device 102 system can be configured to identify an abnormal chewing motion. In various embodiments, the abnormal chewing motion can include an offset chewing motion. Detection of an offset chewing motion can be taken as an indication of a dental health condition.

In some cases, dental health issues may be related to or may otherwise impact breathing. Breathing results in characteristic sounds and/or movement that can be identified within the signals for sensors herein including, for example, within microphone and/or movement sensor signals. As such, in various embodiments, the car-wearable device 102 system can be further configured to detect breathing including abnormal breathing. In various embodiments, the abnormal breathing can include at least one including at least one of snoring and sleep apnea. As an example, snoring sounds can be distinguished from normal breathing sounds based on sound volume, with snoring including relatively loud sounds of greater than 45, 50, or 55 dB occurring during periods of sleep. Sleep apnea can be characterized by snoring followed by silent breathing pauses and/or choking or gasping sounds.

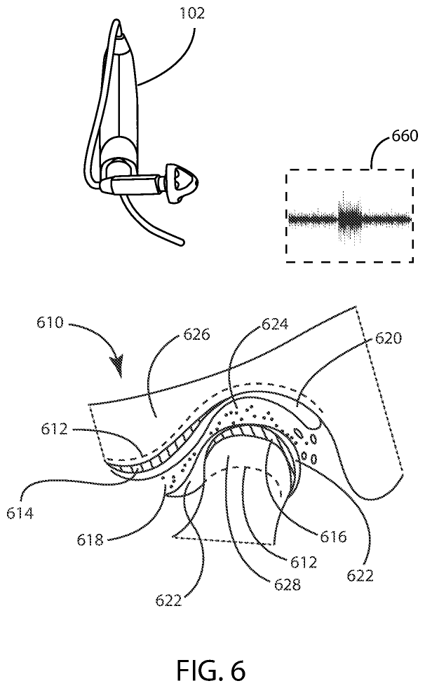

The temporomandibular joint (TMJ) is the small joint in front of the ear where the lower jaw fits in to the base of the skull. Individuals can develop problems with the TMJ joint as a result of injuries, arthritis, genetics, or the like. Systems herein can detect TMJ joint problems. Referring now to FIG. 6, a schematic view of components of an car-wearable device system is shown in accordance with various embodiments herein. In this example, an car-wearable device 102 is shown along with a view of a normal temporomandibular joint (TMJ) 610 in a closed mouth view. In this view, the condyle 628 is seated in its most posterior and superior position. The view as shown is slightly distracted to show detail of the associated upper bearing surface 614 of the articular eminence 626, the articular disk (posterior band 624 and anterior band 618) and the lower bearing surface 616 of the condyle 628. The capsule attachment 612 (shown as a dashed line) is shown for the upper and lower joint. The inner surface of the capsule along with upper joint space 620 and lower joint space 622 forms a lateral recess space. The anterior and posterior recesses of the upper, lower and lateral joint spaces are lined with synovial tissue.

As the mouth opens, the condyle 628 undergoes translation and rotation. In most cases, this occurs without much noise. However, in some cases mouth opening can be associated with popping or clicking within the jaw joint which can be indicative of a TMJ joint problem. Popping or clicking is generally occurs when the articular disk is positioned in front of the condyle 628 and on opening the mouth, the condyle 628 slides on to the articular disk resulting in the noise.

The popping or clicking of the TMJ joint can create a distinctive pattern in a sensor signal 660 herein. Embodiments of systems herein can be used to detect sound and/or motion patterns that are consistent with TMJ joint problems. For example, embodiments of systems herein can be used to detect the characteristic popping or clicking of a TMJ joint problem.

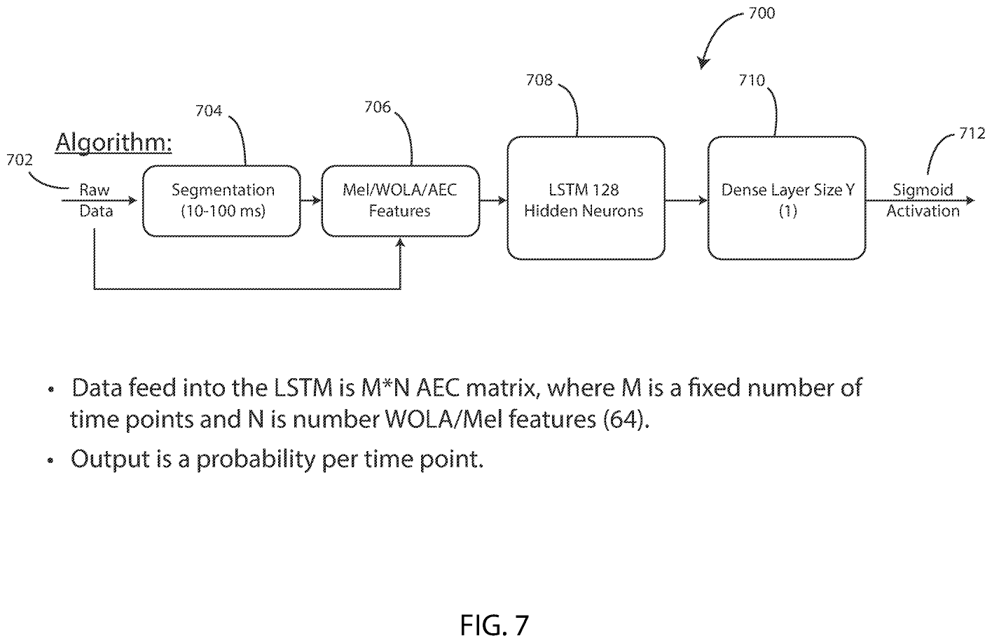

It will be appreciated that systems and/or devices herein can execute various data processing and/or signal processing operations in order to detect things such as dental health conditions, dental health status, and/or dental hygiene events. Referring now to FIG. 7, a diagram of data processing operations 700 is shown in accordance with various embodiments herein. Raw sensor data 702 can be subjected to an operation of segmentation 704. Segmentation can include breaking up the signal into discrete units cover a specific time span. Various time spans can be used, however in some embodiments the time span can be on the order of millisecond, such as 5, 10, 25, 50, 100, 150, 200, 300 milliseconds, or an amount a time falling within a range between any of the foregoing.

The segmented signal data can then be used in operations of feature extraction and signal processing 706. Feature extraction and signal processing 706 herein can include the use of Mel frequency cepstral coefficient (MFCC) feature extraction. Feature extraction and signal processing 706 can also include WOLA (weighted-overlap add) algorithm signal processing creating WOLA bands. Feature extraction and signal processing 706 can also include an acoustic echo cancelation (AEC) operation.

The processed data (such as WOLA bands) can then be evaluated 708 in an LSTM recurrent neural network using a plurality of neuron units. The data fed into the LSTM can by an M*N AEC matrix, where M is a fixed number of time points and N is a number of WOLA/Mel features. In some embodiments, two layers of LSTM are used with 128 hidden layers in the first layer and 256 layers in the second. A supervised learning approach can be used with labeled learning data including recordings of dental health relevant events such as toothbrushing sounds, bruxism sounds, TMJ joint popping, or the like. However, semi-supervised learning approaches can also be used. The LSTM recurrent neural network output can then be fed into a dense layer 710 (a fully connected layer that follows LSTM layers and is used for outputting a prediction). The output of the dense layer can then be subject to a sigmoid activation 712 function as the last layer. The final output can be a probability per time period. For example, the final output can be probability per time period of a dental hygiene event, such as tooth brushing, taking place. In some embodiments, the probability can be compared with a threshold value and if the threshold value is exceeded then the time period or frame can be taken as indicative of the particular dental health related event having occurred. Alternatively, the probability can be related to any dental health condition or dental health status herein. It will be appreciated that the described approach for data processing/signal processing is only one specific example and many variations on this approach are contemplated herein.

Many different types of dental health related events can be detected using data and/or signal processing techniques herein. For example, in some embodiments, data and/or signal processing techniques can be used herein to identify an occurrence of a dental hygiene event. As such, the car-wearable device system can be configured to evaluate signals from at least one of the microphone and the motion sensor to detect an occurrence of a dental hygiene event.

In various embodiments, the dental hygiene event can include at least one including at least one of teeth brushing and teeth flossing. In various embodiments, the car-wearable device system can be configured to track occurrences of dental hygiene events over time. In various embodiments, the car-wearable device system can be configured to track frequencies of dental hygiene events over time. In various embodiments, the car-wearable device system can be configured to track durations of dental hygiene events over time.

In various embodiments, the ear-wearable device system can be configured to prompt a device wearer to undertake the dental hygiene event. For example, a prompt or other notification can be presented to the device wearer audibly, visually, and/or haptically. In some embodiments, the prompt or other notification can be presented to the device wearer directly from the car-wearable device itself. In other embodiments, the prompt or other notification can be presented to the device wearer from an accessory device in electronic communication with the car-wearable device. In some embodiments, a prompt or other notification can be presented to a care provider.

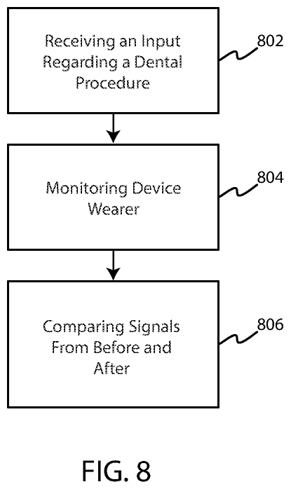

Referring now to FIG. 8, a flowchart of operations performed is shown in accordance with various embodiments herein. The operations of FIG. 8 can serve as an example of operation executed as part of a method of tracking effects of a dental procedure. In various embodiments, an operation of receiving an input regarding a dental procedure 802 can be performed. In some embodiments, the input can be received from an individual such as the device wearer, a care provider, a clinician (such as a dentist), or the like. In some embodiments, the input can be received from another device or system. In some embodiments the input can be received from an electronic medical records system. The input can be generic regarding a dental procedure occurring or can be specific and include information on one or more of the type of dental procedure, which teeth are affected by the dental procedure, the date of the dental procedure, and the like.

In various embodiments, an operation of monitoring 804 the device wearer can be performed. Monitoring 804 can include gathering signal data from sensors as described herein. Data can be gathered continuously or intermittently such as according to a duty cycle. The sensor data can be processed in various ways in order to detect things such as dental health conditions, dental health status, and/or dental hygiene events.

In various embodiments, an operation of comparing signals from at least one of the microphone and the motion sensor from a time period prior to the dental procedure with signals from at least one of the microphone and the motion sensor from a time period after the dental procedure 806 can be performed.

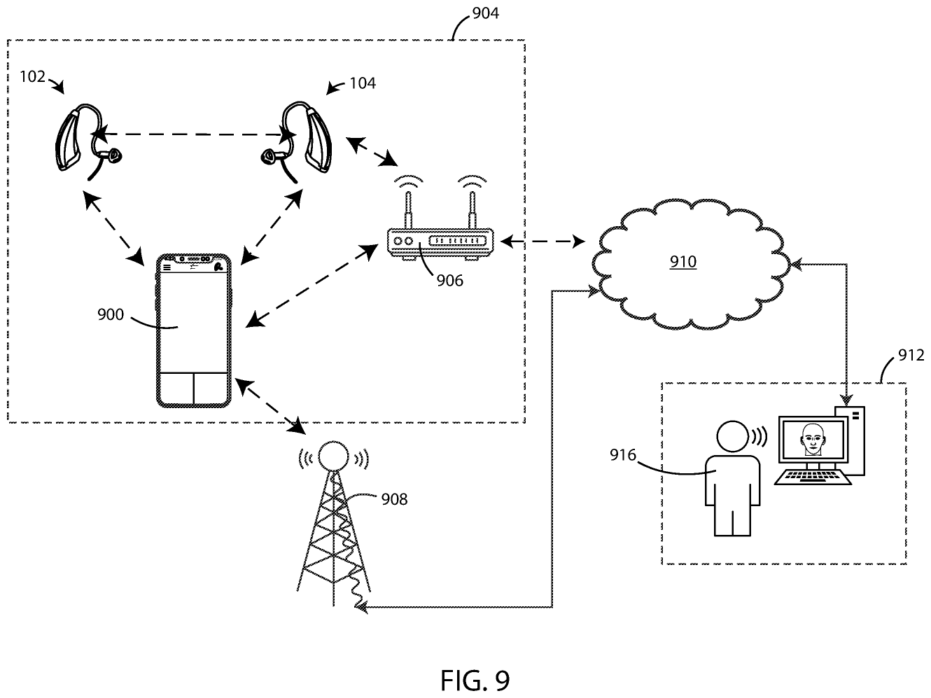

Systems herein can include various components and can be in electronic communication with one another for purposes of exchanging data, sensor signals, notification and/or communications, and the like. Referring now to FIG. 9, a schematic view of components of an ear-wearable system is shown in accordance with various embodiments herein. FIG. 9 shows a first car-wearable device 102 and a second car-wearable device 104 at a first location or device wearer location 904. In some embodiments, the system can include and/or can interface with other devices at the first location 904. The other devices in this example can include an accessory device 900, which could be a smart phone or similar mobile communication/computing device in some embodiments. The accessory device 900 can include sensors and the data and/or signals from the same can be provided to the car-wearable devices herein. In some embodiments operations described herein can be performed on the car-wearable devices themselves. In other embodiments, operations described herein can be performed on the accessory device 900 and/or using a cloud computing resource.

FIG. 9 also shows communication equipment including a cell tower 908 and a network router 906 to facilitate data communication. FIG. 9 also schematically depicts the cloud 910 or similar data communication network and/or remote computing resources. The communication equipment can provide data communication capabilities between the car-wearable devices 102, 104 and other components of the system and/or components such as the cloud 910 and cloud resources such as a cloud computing resource. In some embodiments, the cloud 910 and/or resources thereof can host an electronic medical records system. In some embodiments, the cloud 910 can provide a link to an electronic medical records system.

FIG. 9 also shows a remote location 912. The remote location 912 can be the site of a third party 916, which can be a clinician (such as a dentist or dental technician), care provider, loved one, or the like. The third party 916 can receive notifications and/or alerts regarding the dental health status of the device wearer. For example, in various embodiments the car-wearable device system can be configured to issue an alert or notification regarding detected occurrences of the dental health condition and the same can be sent on to a third party 916. In various embodiments, the car-wearable device system can specifically be configured to issue an alert or notification when a worsening of a dental condition is detected based on the signals from at least one of the microphone and the motion sensor.

In some embodiments, the third party 916 can provide instructions for the device wearer regarding actions to take, such as dental hygiene actions. In various embodiments, the car-wearable system can be configured to send information regarding dental health to an electronic medical record system.

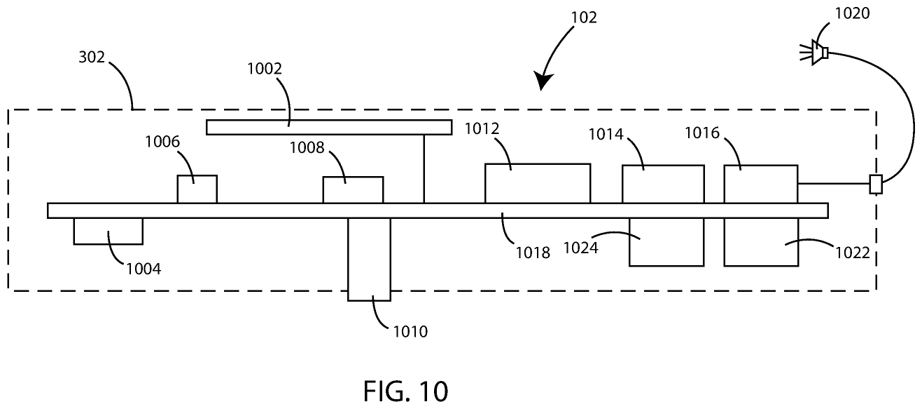

Ear-wearable devices herein can include many different components. Referring now to FIG. 10, a schematic block diagram is shown illustrating various components of an car-wearable device in accordance with various embodiments herein. It will be appreciated that many of these components can be integrated in an integrated circuit, such as with a system-on-a-chip (SOC) integration or can exist as separate components. The ear-wearable device 102 shown in FIG. 10 includes several components electrically connected to a flexible mother circuit 1018 (e.g., flexible mother board) which is disposed within housing 302. A power supply circuit 1004 can include a battery and can be electrically connected to the flexible mother circuit 1018 and provides power to the various components of the car-wearable device 102. One or more microphones 1006 are electrically connected to the flexible mother circuit 1018, which provides electrical communication between the microphones 1006 and a digital signal processor (DSP) 1012. Among other components, the DSP 1012 incorporates or is coupled to audio signal processing circuitry configured to implement various functions described herein. A sensor package 1014 can be coupled to the DSP 1012 via the flexible mother circuit 1018. The sensor package 1014 can include one or more different specific types of sensors such as those described in greater detail below. One or more user switches 1010 (e.g., on/off, volume, mic directional settings) are electrically coupled to the DSP 1012 via the flexible mother circuit 1018.

An audio output device 1016 is electrically connected to the DSP 1012 via the flexible mother circuit 1018. In some embodiments, the audio output device 1016 comprises a speaker (coupled to an amplifier). In other embodiments, the audio output device 1016 comprises an amplifier coupled to an external receiver 1020 adapted for positioning within an ear of a wearer. The external receiver 1020 can include an electroacoustic transducer, speaker, or loudspeaker. The ear-wearable device 102 may incorporate a communication device 1008 coupled to the flexible mother circuit 1018 and to an antenna 1002 directly or indirectly via the flexible mother circuit 1018. The communication device 1008 can be a Bluetooth® transceiver, such as a BLE (Bluetooth® low energy) transceiver or other transceiver(s) (e.g., an IEEE 802.11 compliant device). The communication device 1008 can be configured to communicate with one or more external devices, such as those discussed previously, in accordance with various embodiments. In various embodiments, the communication device 1008 can be configured to communicate with an external visual display device such as a smart phone, a video display screen, a tablet, a computer, a television, a virtual or augmented reality, a hologram, or the like.

In various embodiments, the car-wearable device 102 can also include a control circuit 1022 and a memory storage device 1024. The control circuit 1022 can be in electrical communication with other components of the device. The control circuit 1022 can execute various operations, such as those described herein. The control circuit 1022 can include various components including, but not limited to, a microprocessor, a microcontroller, an FPGA (field-programmable gate array) processing device, an ASIC (application specific integrated circuit), or the like. The memory storage device 1024 can include both volatile and non-volatile memory. The memory storage device 1024 can include ROM, RAM, flash memory, EEPROM, SSD devices, NAND chips, and the like. The memory storage device 1024 can be used to store data from sensors as described herein and/or processed data generated using data from sensors as described herein.

In various embodiments, a spatial location determining circuit (or geolocation circuit) can be included and can take the form of an integrated circuit that can include components for receiving signals from GPS, GLONASS, BeiDou, Galileo, SBAS, WLAN, BT, FM, and/or NFC type protocols.

### Methods

Many different methods are contemplated herein, including, but not limited to, methods of making, methods of using, methods of monitoring dental health, methods of detecting dental health issues, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of detecting dental health conditions using an ear-wearable system is included. The method can include monitoring a device wearer with sensors of the ear-wearable system, the sensors can include at least one of a microphone and a motion sensor. The method can further include evaluating signals from at least one of the microphone and the motion sensor to detect an occurrence of a dental health condition. The method can further include issuing an alert or notification regarding detected occurrences of the dental health condition. In an embodiment, the dental health condition can include at least one selected from the group consisting of bruxism, a TMJ joint issue, oral pain, and tooth decay.

In an embodiment, the method can further include time stamping detected occurrences of dental health conditions and identifying time-related patterns of the same.

In an embodiment, the method can further include distinguishing between awake bruxism and sleep bruxism.

In an embodiment, the method can further include identifying differences in dental health conditions between a left side of the mouth and a right side of the mouth. In an embodiment, the method can further include identifying differences in sounds originating from a left side of the mouth versus a right side of the mouth. In an embodiment, the method can further include distinguishing between left sided TMJ joint problems and right sided TMJ joint problems.

In an embodiment, the method can further include detecting abnormal breathing. In an embodiment, the abnormal breathing can include at least one selected from the group consisting of snoring and sleep apnea.

In an embodiment, a method of tracking effects of a dental procedure is included. The method can include receiving an input regarding a dental procedure. The method can further include monitoring a device wearer with sensors of the ear-wearable system, the sensors can include at least one of a microphone and a motion sensor. The method can further include comparing signals from at least one of the microphone and the motion sensor from a time period prior to the dental procedure with signals from at least one of the microphone and the motion sensor from a time period after the dental procedure. In an embodiment, the method can further include issuing an alert or notification when a worsening of a dental condition is detected based on the signals from at least one of the microphone and the motion sensor.

In an embodiment, a method of detecting dental hygiene events is included. The method can include monitoring a device wearer with sensors of the ear-wearable system, the sensors can include at least one of a microphone and a motion sensor. The method can further include evaluating signals from at least one of the microphone and the motion sensor to detect an occurrence of a dental hygiene event.

In an embodiment, the method can further include tracking occurrences of dental hygiene events over time. In an embodiment, the method can further include tracking frequencies of dental hygiene events over time. In an embodiment, the method can further include tracking durations of dental hygiene events over time. In an embodiment, the method can further include timing stamp detected occurrences of dental hygiene events and identify time-related patterns of the same.

In an embodiment, the method can further include prompting the device wearer to undertake the dental hygiene event.

### Sensors

Ear-wearable devices and/or systems herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, filed Oct. 21, 2016, which is incorporated herein by reference. As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

In some embodiments, the motion sensors can be disposed in a fixed position with respect to the head of a device wearer, such as worn on or near the head or ears. In some embodiments, the operatively connected motion sensors can be worn on or near another part of the body such as on a wrist, arm, or leg of the device wearer.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system

(GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, spatial location sensors and/or geolocation sensors can be included and can take the form of an integrated circuit that can include components for receiving signals from GPS, GLONASS, BeiDou, Galileo, SBAS, WLAN, BT, FM, and/or NFC type protocols

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso).

### Pattern Identification and Pattern Matching

It will be appreciated that in various embodiments herein, a device or a system can detect dental health conditions, track a device wearer's recovery and/or progress after a dental procedure, detect occurrences of dental hygiene events, and the like of a device wearer by evaluating data and identifying a pattern regarding the same and/or matching the data against patterns or templates that are indicative of specific dental health conditions, dental health statuses, dental hygiene events, or the like. Patterns can be identified and/or matched in various ways. As merely one example, one or more sensors can be operatively connected to a controller (such as the control circuit describe in FIG. 10) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning.

Further, in some embodiments, the controller can be adapted to compare data, data features, and/or statistics against various other patterns, which could be prerecorded patterns (baseline patterns) of the particular individual wearing an car-wearable device herein, prerecorded patterns (group baseline patterns) of a group of individuals wearing car-wearable devices herein, one or more predetermined patterns that serve as patterns indicative of specific dental health conditions, dental health statuses, or dental hygiene events (positive example patterns), one or more predetermined patterns that service as patterns indicative of the absence of specific dental health statuses (negative example patterns), or the like. As merely one scenario, if a pattern is detected in an individual that exhibits similarity crossing a threshold value to a positive example pattern or substantial similarity to that pattern, then that can be taken as an indication of the individual having the dental health condition, dental health status, or dental hygiene event associated with the example pattern.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with dental health condition, dental health status, or dental hygiene event of an individual (and, in particular, their status with respect to an orthopedic status), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

### Own Voice/Sound Detection—Sound Location Detection

Distinguishing between speech or sounds associated with the device wearer and speech or sounds associated with a third party can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same car-wearable device or on different car-wearable devices) this can be done through spatial localization of the origin of the speech or other sounds and filtering out, spectrally subtracting, or otherwise discarding sounds that do not have an origin within the device wearer. In some embodiments, such as where there are two or more car-worn devices, own-voice detection can be performed and/or enhanced through correlation or matching of intensity levels and or timing.

In some cases, the system can include a bone conduction microphone to preferentially pick up sounds of the device wearer. In some cases, the system can include a directional microphone that is configured to preferentially pick up sounds of the device wearer. In some cases, the system can include an intracanal microphone (a microphone configured to be disposed within the car-canal of the device wearer) to preferentially pick up sounds of the device wearer. In some cases, the system can include a motion sensor (e.g., an accelerometer configured to be on or about the head of the wearer) to preferentially pick up skull vibrations associated with sounds of the device wearer.

In some cases, an adaptive filtering approach can be used. By way of example, a desired signal for an adaptive filter can be taken from a first microphone and the input signal to the adaptive filter is taken from the second microphone. If the device wearer is making sounds, the adaptive filter models the relative transfer function between the microphones. Own-voice/sound detection can be performed by comparing the power of an error signal produced by the adaptive filter to the power of the signal from the standard microphone and/or looking at the peak strength in the impulse response of the filter. The amplitude of the impulse response should be in a certain range to be valid for their own voice/sound. If the user's own voice/sound is present, the power of the error signal will be much less than the power of the signal from the standard microphone, and the impulse response has a strong peak with an amplitude above a threshold. In the presence of the user's own voice/sound, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, the system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F₀. A voice/sound detection circuit is adapted to receive the second output signal B and the pitch output F₀ and to produce an own voice/sound detection trigger T. The apparatus further includes a mismatch filter adapted to receive and process the second output signal B, the own voice/sound detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In various embodiments herein, a device or system can specifically include one or more inward-facing microphones (e.g., facing the car canal, or facing tissue, as opposed to facing the ambient environment.) A sound signal captured by the inward-facing microphone can be used to determine physiological information, such as that relating to a dental health condition, dental health status, or dental hygiene event or a device wearer.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device system comprising:
an ear-wearable device, the ear-wearable device comprising
a control circuit;
a microphone, wherein the microphone is in electrical communication with the control circuit; and
a sensor package, the sensor package comprising a motion sensor;
wherein the sensor package is in electrical communication with the control circuit; and
wherein the ear-wearable device system is configured to evaluate signals from at least one of the microphone and the motion sensor to detect an occurrence of a dental health condition.

2. The ear-wearable device system of claim 1, the dental health condition comprising at least one selected from the group consisting of bruxism, a TMJ joint issue, oral pain, and tooth decay.

3. The ear-wearable device system of claim 1, wherein the ear-wearable device is a hearing-assistance device.

4. The ear-wearable device system of claim 1, wherein the ear-wearable device system is configured to issue an alert or notification regarding detected occurrences of the dental health condition.

5. The ear-wearable device system of claim 1, wherein the ear-wearable device is configured to time stamp detected occurrences of dental health related conditions and identify time-related patterns of the same.

6. The ear-wearable device system of claim 5, wherein the ear-wearable device is configured to identify sleep bruxism.

7. The ear-wearable device system of claim 1, wherein the ear-wearable device system is configured to distinguish between awake bruxism and sleep bruxism.

8. The ear-wearable device system of claim 1, further comprising:
a second ear-wearable device, the second ear-wearable device comprising
a second control circuit;
a second microphone; and
a second sensor package; and
wherein the second ear-wearable device is configured to be wearable on an opposite ear versus the ear-wearable device.

9. The ear-wearable device system of claim 8, wherein the ear-wearable device system is configured to identify differences in dental health related conditions between a left side of the mouth and a right side of the mouth.

10. The ear-wearable device system of claim 8, wherein the ear-wearable device system is configured to identify an abnormal chewing motion.

11. The ear-wearable device system of claim 10, the abnormal chewing motion comprising an offset chewing motion.

12. The ear-wearable device system of claim 8, wherein the ear-wearable device system is configured to identify differences in sounds originating from a left side of the mouth versus a right side of the mouth.

13. The ear-wearable device system of claim 12, wherein the ear-wearable device system is configured to distinguish between left sided TMJ joint problems and right sided TMJ joint problems.

14. The ear-wearable device system of claim 1, wherein the ear-wearable device system is further configured to detect abnormal breathing.

15. The ear-wearable device system of claim 14, the abnormal breathing comprising at least one selected from the group consisting of snoring and sleep apnea.

16-24. (canceled)

25. An ear-wearable device system comprising:
an ear-wearable device, the ear-wearable device comprising
a control circuit;
a microphone, wherein the microphone is in electrical communication with the control circuit; and
a sensor package, the sensor package comprising a motion sensor;
wherein the sensor package is in electrical communication with the control circuit; and
wherein the ear-wearable device system is configured to compare signals from at least one of the microphone and the motion sensor from a time period prior to a dental procedure with signals from at least one of the microphone and the motion sensor from a time period after the dental procedure.

26. The ear-wearable device system of claim 25, the dental procedure comprising at least one selected from the group consisting of a dental implant, a crown, an orthodontic adjustment, and a tooth filling.

27. The ear-wearable device system of claim 26, wherein the ear-wearable device system is configured to receive an input regarding a type of dental procedure from a system user.

28. The ear-wearable device system of claim 25, wherein the ear-wearable device system is configured to issue an alert or notification when a worsening of a dental condition is detected based on the signals from at least one of the microphone and the motion sensor.

29. (canceled)

30. The ear-wearable device system of claim 25, further comprising:
a second ear-wearable device, the second ear-wearable device comprising
a second control circuit;
a second microphone; and
a second sensor package; and
wherein the second ear-wearable device is configured to be wearable on an opposite ear versus the ear-wearable device.

31-53. (canceled)
